(19) United States
(12) Patent Application Publication
(10) Pub. No.: US 2026/0117645 A1
(43) Pub. Date: Apr. 30, 2026

(54) METHOD OF USING TRACERS FOR PROVIDING A DATA SET RELATED TO WELLBORE PERFORMANCE

(71) Applicant: Talgat Shokanov, Houston, TX (US)

(72) Inventors: Talgat Shokanov, Houston, TX (US); Quanxin Guo, Sugar Land, TX (US); John Oliver, Sugar Land, TX (US); Francisco Fragachan, Katy, TX (US)

(21) Appl. No.: 19/376,243

(22) Filed: Oct. 31, 2025

Related U.S. Application Data
(63) Continuation-in-part of application No. 18/953,826, filed on Nov. 20, 2024.
Application No. 18/953,826 is a continuation-in-part of application No. 17/688,316, filed on Mar. 7, 2022, now abandoned.

(51) Int. Cl. E21B 47/11; G01N 23/223; G01N 33/28

(52) U.S. Cl. CPC E21B 47/11; G01N 23/223; G01N 33/2823; G01N 2223/637

(57) ABSTRACT

A method of using a tracer in a wellbore that includes disposing the tracer into the wellbore so that the tracer comes into contact with a target formation. Upon contacting the target formation for an amount of time, returning a remnant fluid that includes at least a portion of the tracer to a surface for testing. The tracer has a first composition, and in its original state is a solid non-magnetic powder.

### BACKGROUND

### FIELD OF THE DISCLOSURE

This disclosure generally relates to the use of an innovative type of chemical additive known as a ‘tracer’ in a wellbore, or other formations such as a subsurface reservoir. The tracer may be solid nanoparticle. The tracer may be disposed into the wellbore in number of ways, and flown out (recovered). A resultant produced fluid may then be tested in manner that facilitates determination of flow performance, inter-well communication, or a model of one or more production parameters associated with the wellbore, created hydraulic fractures, and/or reservoir production performance. The test may provide a data set usable to establish an indication of quality associated with the wellbore/reservoir. The data set may be used to provide an indication of reservoir quality, completion quality, or both.

### BACKGROUND OF THE DISCLOSURE

A hydrocarbon-based economy and an emerging geothermal power supply continue to be dominant force in the modern world. As such, locating and producing hydrocarbons continues, along with understanding the flow performance of subsurface formations, demand attention from the oil and gas (O&G) industry. A well or wellbore is generally drilled in order to recover valuable hydrocarbons and other desirable materials trapped in geological formations in the Earth, which are later refined into commercial products, such as gasoline or natural gas.

A wellbore is typically drilled using a drill bit attached to the lower end of a “drill string.” Once the drilling is finished, a production string is typically placed all the way into the wellbore. To gain access to hydrocarbons, selected portions of the production string (and formation) are often perforated. Common today to increase or enhance production in the tight or unconventional reservoirs is the use of multistage hydraulic fracturing (i.e., “fracing”) in the surrounding formations.

Fracing entails the pumping of fracturing fluids with sand into a formation in an open-hole or via perforations in a cased wellbore or other openings in the casing to form a fracture(s) in the formation. Fracing routinely requires very high fluid pressure and pumping rate and can occur in a multistage fracing manner. The well construction design may entail an open hole, cased hole, lined hole, etc.

The modern design of shale well with multi-stage hydraulic fracturing operations involve pumping from 20 to 100 fracing stages with a cumulative volume of 5 to 20 million gallons of water and from 5 to 20 million pounds of sand per well. This represents the total cost ranging from 4.0 million to 9.5 million U.S. dollars per well.

Approximately 7,000 horizontal wells were drilled, and 250,000 stages completed in North America alone in 2020. Additionally, current multi-stage fracing operations are still expensive, increasingly environmentally challenging and emissions intensive. Multi-stage fracing operations already represent up to 70% of the total cost for each well. If current trends of increasing horizontal lateral length and adding more stages per well continue using current brute force approach, it is estimated that up to 25% of new wells will be uneconomical.

Fracing or other forms of production stimulation methods such as Improved Oil Recovery (IOR) and Enhanced Oil Recovery (EOR) are typically used in either conventional or unconventional wellbores. The difference between what is commonly understood as conventional or unconventional relates to rock permeability, or rather, how tight the rock/formation is. Unconventional wells also tend to have vast and unpredictable formation variation (reservoir quality), and receive less attention, as profitability is often reduced or limited as a result of higher costs associated with well construction, fracing, and fast production decline.

In the event of dealing with an unconventional (or even conventional) well, production diagnostic tools may be used in order to predict well performance, improve well design, or aid in future well development. Typically, diagnostic or surveillance tools include fiber, PLT (production logging), fiber-optic, and liquid chemical tracers.

Use of fiber optic systems that include distributed acoustic sensing (DAS) and distributed temperature surveys (DTS) is known to provide high-end diagnostic results. However, fiber is known to be excessive in cost and deployment complexities, and the time to obtain useful data may be in the realm of weeks or longer. Depending on the complexity, the installation of fiber optic DAS and DTS systems can add as much as $1 million/well to the completed total costs.

PLT also has its favored uses and is a historically well accepted approach, but while perhaps slightly lower in cost, it is known to provide a very short snapshot view and information compared to fiber and requires well shut-in and costly wireline intervention.

Conventional chemical liquid tracers that are dissolvable in oil or water have enjoyed success but are also known to have limitations. These tracers are dissolvable in oil and water phases, and typically have fluorescent properties, DNA and ionic, organic materials, or radioactive diagnostic isotopes. Such tracers are used to evaluate fracturing performance, ostensibly to control the effectiveness of multi-stage hydraulic fracturing stimulation. Owing to obvious environmental deficiencies, tracers incorporating radioactive isotopes have largely fallen out of favor. Given their soluble characteristics, conventional chemical tracers must be tailored for individual fluid types, thereby requiring more, and often exotic, formulations for a single stage, increasing the chemical tracer costs appreciably.

Given the inherent heterogeneity of the rock along a typical horizontal lateral (i.e., horizontal wellbore) and the assorted fluid streams, the different types of liquid chemical tracers required could add up significantly in incremental costs/well. Furthermore, once liquid-based tracers have been pumped, they disseminate quickly and flush from the proppant pack, shortening the effective monitoring period significantly. Thus, between occasionally inconclusive accuracy, cross-well contamination, and downhole temperature restrictions (limited to 400 F), the use of contemporary liquid tracers is limited.

In the same vein, conventional tracer testing is severely restricted by the time required to obtain a comprehensive interpretation of the test results. This is normally accomplished from an offsite lab with a minimum three-week turnaround on average, given the longer sample preparation time, very expensive instrumentation, sensitive samples dissolution process and specialized argon gas and reagents needed for analysis

Perhaps one of the more glaring drawbacks with liquid tracers is the limitation to only chemical measurement techniques at a molecular level, and the frequent instigation of unnecessary signals to what is erroneously perceived as “frac-hits”. A frac hit is typically described as a fracture-driven inter-well communication event where an offset well, often termed a parent well in this setting, is affected by the pumping of a frac treatment in a new well, called the child well.

Each of the aforementioned techniques: fiber, PLT, and liquid chemical tracer tools have temperature limitations (i.e., for use in <700° F.) that make their use problematic at best in unconventional reservoirs as well as geothermal formations, where temperatures may be as high as 800° F.

Moreover, reducing emissions and environmental footprint from multi-stage hydraulic fracturing operations is a high-priority metric in oil and gas, as operators staunchly embraced environmental, social and governance (ESG) initiatives. In addition, fracture-driven interactions between fractures of the new wells (i.e., child wells) with adjacent horizontal wells (i.e., parent wells) and their costly negative effects have become the focus of much discussion and debate within the technical community. The negative impact of these frac-to-frac interactions on well productivity, including a rapid drop in production, poor well economics and the mechanical integrity of these parent wells, was the driving force behind such attention.

Intensified focus on the production performance of unconventional wells, combined with the complexities of subsurface reservoir quality and stage-level production, has significantly heightened the economic pressures on oil and gas companies. In this challenging and dynamic environment, operators face increased demands to gain deeper insights into complex reservoir performance, optimize well spacing, and implement data-driven stimulation designs based on stage-level production measurements, all to boost production flow while adhering to increasingly tight budget constraints.

With current completion designs, the production results of multi-stage hydraulic fracturing treatments, when reported down to the individual stage level, are significantly lower than many oil and gas companies expect.

Conventional tracer testing is also significantly limited by the time required to achieve a comprehensive interpretation of the test results. Typically, this process is conducted by an offsite analytical laboratory, which usually has a minimum turnaround time of three weeks on average. This extended timeline is due to lengthy sample preparation procedures, the high cost of instrumentation, the delicate sample dissolution process, and the specialized reagents required for analysis.

The industry needs a low-cost, stage-by-stage flow profiling method that can be used for assessing unconventional reservoirs quality, the completion designs, completion quality, and to advance the multi-stage fracing diagnostics to the next level.

Thus, there is an urgent need to have accurate, affordable, timely data on the performance of individual stages, measured intra-well and frac-to-frac communication. What is needed is a new and improved way of forming and using a fast, cost-favorable, effective, and reliable way of predicting and validating wellbore performance.

There is a need in the art for wellbore development and completion designs validated by cost-effective and accurate flow mapping measurements that deepen insight into complex reservoir flow performance and geological risk assessment, right-size the multi-bench well spacing, and focus on data-driven stimulation designs that optimize economics.

The need for a solid tracer that is versatile, affordable, highly accurate, non-radioactive, non-intrusive and quick to test is increasing as never before for all subsurface, production and injection applications.

### SUMMARY

Embodiments of the disclosure pertain to a method of using a tracer additive (or sometimes just ‘tracer’) in a wellbore that may include one or more steps described herein.

The method may include disposing one or more tracers into a wellbore. Any tracer may have a unique identifier or marker. A first specific, identifiable tracer may be disposed into a first targeted stage of the wellbore. A second specific, identifiable tracer may be disposed into a second targeted stage of the wellbore. There may be a plurality of specific, identifiable tracers disposed into respective targeted stages of the wellbore.

Disposing any tracer into the wellbore may occur via one or more techniques, such as mixing the tracer with a pumpdown or utility fluid, and flowing that mixture into the wellbore. The method may include using a dissolving device that need not be connected with a tubestring, and thus may be flown into the wellbore. In other aspects, the dissolving device may be a downhole tool coupled with the tubestring.

The disposing step may occur in such a manner that the tracer may eventually come into contact with a target formation. The target formation may be a stage. The target formation may be in (fluid) communication with the wellbore

Upon contacting the target formation for an amount of time, the method may include returning a remnant fluid to a surface or surface facility. The produced remnant fluid may include at least a portion of the tracer(s). The produced remnant fluid may come from the same wellbore as the disposing step, or another wellbore.

The method may include taking a sample of the remnant fluid. In that event, the method may include testing the sample in order to analyze the remnant fluid. At this point this may result in obtaining or otherwise providing a set of (fluid) data associated therewith. The method may include integrating the set of fluid data with other wellbore data in order to determine a parameter associated with performance of the wellbore.

In aspects, the tracer may have a first tracer composition. In other aspects, the tracer may be in a solid powder form. The powder may have an average particle diameter of at least 0.1 μm to no more than 10 μm. In aspects, the average particle diameter may be at least 0.1 μm to less than 1 μm. The tracer may have an average bulk specific gravity of at least 0.6 to no more than 1.2.

The wellbore or target formation may have various parameters associated with it. For example, the wellbore or target formation may be associated with a formation temperature of at least 1000° F. to no more than 2000° F. In other aspects, the wellbore or target formation may have an average permeability of 0.1 nanodarcy to 1000 nanodarcy.

The target formation may be part of a geothermal well, EOR process, or horizontal drilled well associated with hydraulic fracturing. In aspects, the remnant fluid may be used in an energy generation process. For example, a fluid may be injected into the geothermal well, energy (such as heat) added thereto, and then the fluid is produced to the surface, where the added energy may be converted in the energy generation process.

In some embodiments, the method may include disposing a second tracer into the wellbore. This may be done in a manner so that the second tracer comes into contact with one or more of the target formation, another target formation proximate to the wellbore, or combinations thereof.

The second tracer may have a different composition from the chemical additive (or first tracer). The second tracer may be in powder form. The second tracer may have an average particle diameter of at least 0.1 μm to no more than 10 μm (or any range inbetween). The second tracer may have an average bulk specific gravity of at least 0.6 to no more than 1.2. Although not limited, the second tracer may be disposed into and produced from the same wellbore.

The target formation may be associated with a frac stage. The wellbore or target formation may be associated with a formation temperature of at least 450° F. The formation temperature may be no more than 2000° F.

In aspects, the testing the sample step comprises using a fluorescence response-based analysis. The fluorescence response-based analysis may include EDXRF. The fluorescence response-based analysis may include XRD. The parameter may be associated with flow mapping of the wellbore.

In aspects, the first tracer, the second tracer, and/or other tracer may be completely non-magnetic (that is, one of ordinary skill in the art would recognize the tracer has having a physical property of being non-magnetic). Any method of the disclosure may be completely void of a magnet(s) or any type of magnetic feature.

The remnant fluid may be filtered prior to testing or analysis. For example, the method may include a filtering step, which may include membrane filtration.

These and other embodiments, features and advantages will be apparent in the following detailed description and drawings.

### BRIEF DESCRIPTION OF THE DRAWINGS

A full understanding of embodiments disclosed herein is obtained from the detailed description of the disclosure presented herein below, and the accompanying drawings, which are given by way of illustration only and are not intended to be limitative of the present embodiments, and wherein:

FIG. 1 shows a side view of a system for using a tracer in a wellbore according to embodiments of the disclosure;

FIG. 1A shows a side view of a system for using a deployment device for the transfer of a tracer into a wellbore according to embodiments of the disclosure;

FIG. 1B shows a side view of a system for using a downhole tool for the transfer of a tracer into a wellbore according to embodiments of the disclosure;

FIG. 1C shows a side view of a system for using a perforator device for the transfer of a tracer into a wellbore according to embodiments of the disclosure;

FIG. 2 is a side view of the system of FIG. 1 where a remnant fluid with the tracer is produced from the wellbore according to embodiments of the disclosure;

FIG. 3 is a simplified block diagram of an analytical unit used to test a sample having a tracer according to embodiments of the disclosure;

FIG. 4 is a side view of the system of FIG. 1 for using a second tracer in a wellbore according to embodiments of the disclosure;

FIG. 5 is a side view of a system for using a tracer in a formation having multiple wellbores according to embodiments of the disclosure;

FIG. 6A shows a side view of a system for using a tracer in a geothermal well according to embodiments of the disclosure;

FIG. 6B shows a side view of the system of FIG. 6A where a remnant fluid with the tracer produced from the wellbore according to embodiments of the disclosure; and

FIG. 6C shows a side view of the system of FIG. 6A where a remnant fluid with the tracer is produced from a proximate wellbore according to embodiments of the disclosure.

### DETAILED DESCRIPTION

Regardless of whether presently claimed herein or in another application related to or from this application, herein disclosed are novel apparatuses, units, systems, and methods that pertain to use of solid tracer additives, details of which are described herein.

Embodiments of the present disclosure are described in detail with reference to the accompanying Figures. In the following discussion and in the claims, the terms “including” and “comprising” are used in an open-ended fashion, such as to mean, for example, “including, but not limited to . . . ”. While the disclosure may be described with reference to relevant apparatuses, systems, and methods, it should be understood that the disclosure is not limited to the specific embodiments shown or described. Rather, one skilled in the art will appreciate that a variety of configurations may be implemented in accordance with embodiments herein.

Although not necessary, like elements in the various figures may be denoted by like reference numerals for consistency and ease of understanding. Numerous specific details are set forth in order to provide a more thorough understanding of the disclosure; however, it will be apparent to one of ordinary skill in the art that the embodiments disclosed herein may be practiced without these specific details. In other instances, well-known features have not been described in detail to avoid unnecessarily complicating the description. Directional terms, such as “above,” “below,” “upper,” “lower,” “front,” “back,” etc., are used for convenience and to refer to general direction and/or orientation, and are only intended for illustrative purposes only, and not to limit the disclosure.

Connection(s), couplings, or other forms of contact between parts, components, and so forth may include conventional items, such as lubricant, additional sealing materials, such as a gasket between flanges, PTFE between threads, and the like. The make and manufacture of any particular component, subcomponent, etc., may be as would be apparent to one of skill in the art, such as molding, forming, press extrusion, machining, or additive manufacturing. Embodiments of the disclosure provide for one or more components to be new, used, and/or retrofitted to existing machines and systems.

Various equipment may be in fluid communication directly or indirectly with other equipment. Fluid communication may occur via one or more transfer lines and respective connectors, couplings, valving, piping, and so forth. Fluid movers, such as pumps, may be utilized as would be apparent to one of skill in the art.

Numerical ranges in this disclosure may be approximate, and thus may include values outside of the range unless otherwise indicated. Numerical ranges include all values from and including the expressed lower and the upper values, in increments of smaller units. As an example, if a compositional, physical or other property, such as, for example, molecular weight, viscosity, melt index, etc., is from 100 to 1,000. it is intended that all individual values, such as 100, 101, 102, etc., and sub ranges, such as 100 to 144, 155 to 170, 197 to 200, etc., are expressly enumerated. It is intended that decimals or fractions thereof be included. For ranges containing values which are less than one or containing fractional numbers greater than one (e.g., 1.1, 1.5, etc.), smaller units may be considered to be 0.0001, 0.001, 0.01, 0.1, etc. as appropriate. These are only examples of what is specifically intended, and all possible combinations of numerical values between the lowest value and the highest value enumerated, are to be considered to be expressly stated in this disclosure. Numerical ranges are provided within this disclosure for, among other things, the relative amount of reactants, surfactants, catalysts, etc. by itself or in a mixture or mass, and various temperature and other process parameters.

### Terms

The term “connected” as used herein may refer to a connection between a respective component (or subcomponent) and another component (or another subcomponent), which can be fixed, movable, direct, indirect, and analogous to engaged, coupled, disposed, etc., and can be by screw, nut/bolt, weld, and so forth. Any use of any form of the terms “connect”, “engage”, “couple”, “attach”, “mount”, etc. or any other term describing an interaction between elements is not meant to limit the interaction to direct interaction between the elements and may also include indirect interaction between the elements described.

The term “fluid” as used herein may refer to a liquid, gas, slurry, single phase, multi-phase, pure, impure, etc. and is not limited to any particular type of fluid such as hydrocarbons.

The term “utility fluid” as used herein may refer to a fluid used in connection with any fluid disposed into a wellbore (akin to an injection fluid). The utility fluid may be pressurized, and may be used to carry an additive into the wellbore. ‘Utility fluid’ may also be referred to and interchangeable with ‘service fluid’ or comparable.

The term “fluid connection”, “fluid communication,” “fluidly communicable,” and the like, as used herein may refer to two or more components, systems, etc. being coupled whereby fluid from one may flow or otherwise be transferrable to the other. The coupling may be direct, indirect, selective, alternative, and so forth. For example, valves, flow meters, pumps, mixing tanks, holding tanks, tubulars, separation systems, and the like may be disposed between two or more components that are in fluid communication.

The term “pipe”, “conduit”, “line”, “tubular”, or the like as used herein may refer to any fluid transmission means, and may be tubular in nature.

The term “tubestring” or the like (such as ‘workstring’) as used herein may refer to a tubular (or other shape) that may be run into a wellbore. The tubestring may be casing, a liner, production tubing, combinations, and so forth. The tubestring may be multiple pipes (and the like) coupled together. The tubestring may be used for transfer of fluids, or used with some other kind of action, such as drilling, running a tool, or any other kind of downhole action, and combinations thereof.

The term “composition” or “composition of matter” as used herein may refer to one or more ingredients, components, constituents, etc. that make up a material (or material of construction). Composition may refer to a flow stream of one or more chemical components.

The term “chemical” as used herein may analogously mean or be interchangeable to material, chemical material, ingredient, component, chemical component, element, substance, compound, chemical compound, molecule(s), constituent, and so forth and vice versa. Any ‘chemical’ discussed in the present disclosure need not refer to a 100% pure chemical. For example, although ‘water’ may be thought of as H2O, one of skill would appreciate various ions, salts, minerals, impurities, and other substances (including at the ppb level) may be present in ‘water’. A chemical may include all isomeric forms and vice versa (for example, “hexane”, includes all isomers of hexane individually or collectively).

The term “water” as used herein may refer to a pure, substantially pure, and impure water-based stream, and may include wastewater, process water, fresh water, seawater, produced water, slop water, treated variations thereof, mixes thereof, etc., and may further include impurities, dissolved solids, ions, salts, minerals, and so forth. Water for a frac fluid can also be referred to as ‘frac water’.

The term “impurity” as used herein may refer to an undesired component, contaminant, etc. of a composition. For example, a mineral or an organic compound may be an impurity of a water stream.

The term “frac fluid” as used herein may refer to a fluid injected into a well as part of a frac operation. Frac fluid is often characterized as being largely water, but with other constituents such as proppant, friction reducers, and other additives or compounds.

The term “produced fluid”, “production fluid”, and the like as used herein may refer to water, gas, mixtures, and the like recovered from a subterranean formation or other area near the wellbore. Produced fluid may include hydrocarbons or aqueous, such as flowback water, brine, salt water, or formation water. Produced water may include water having dissolved and/or free organic materials. Produced fluid may be akin to ‘wellbore fluid’, in that the fluid may be returned from the wellbore. Produced fluid may include utility fluids and formation fluids.

The term “frac operation” as used herein may refer to fractionation of a downhole well that has already been drilled. ‘Frac operation’ can also be referred to and interchangeable with the terms fractionation, hydraulic fracturing, well stimulation, production enhancement, hydrofracturing, hydrofracking, fracking, fracing, and frac. A frac operation can be land or water based. Generally, the term ‘fracing’ or ‘frac’ is used herein, but meant to be inclusive to other related terms of industry art.

The phrase “processing a fluid” as used herein may refer to some kind of active step or action, such as man-made or by machine, imparted on the fluid (or fluids). For example, a fluid may be received into a device (such as a mixer) and upon processing, may leave as a ‘processed fluid’. ‘Processed’ is not meant be limited, as this may include reference to transferred, treated, tested, measured, mixed, sensed, separated, combinations, etc. in whatever manner may be desired or applicable for embodiments herein. It is noted that while various steps or operations of any embodiment herein may be described in a sequential manner, such steps or operations may be operated in batch or continuous fashion.

The term “conventional well” as used herein may refer to a subterranean formation having an average permeability in the magnitude range of a millidarcy or higher.

The term “unconventional well” as used herein may refer to a subterranean formation having an average permeability in the magnitude range of a nanodarcy or smaller.

The term “tracer” as used herein may refer to an identifiable substance, such as a liquid dye, liquid chemical or a particles powder, which may be followed through the course of a mechanical, chemical, or biological process. In the present disclosure, a tracer may be used in a well, and the resultant process impact on the tracer evaluated. In this respect, the tracer may help evaluate, determine, and otherwise model well production and performance. The tracer may be added (and thus may be referred to as a ‘tracer additive’ or ‘additive’) to a utility (or service, injection, etc.) fluid disposed into the well.

The term “dispose” as used herein may refer to a placement or positioning, which need not be done manually. Those a machine or tool may be used dispose something. For example, disposing a tracer into a wellbore is something that may be accomplished via use of a pump and a pumpable fluid. Dispose may be analogous to dispense, disperse, place, position, etc. or causation for the same.

The term “nanoparticle” as used herein may refer to a small particle that ranges between 1 to 1000 nanometers in size diameter, and is undetectable by the human eye. A tracer in powder form may be nanoparticles. A tracer of the present disclosure may be in powder form with an average bulk diameter in a range of about 0.1 μm to about 10 μm (or any range therebetween). A range therebetween may be at least 0.1 μm to less than 1 μm.

The term “EDXRF” (Non-destructive Energy Dispersive X-Ray Fluorescence) as used herein may refer to a type of spectroscopy process (and may thus include use of a spectrometer) where a sample of material (such as a portion of produced fluid) is ‘excited’ in order to collect emitted fluorescence radiation, which may then be evaluated for different energies of the characteristic radiation from each of the different constituents (or elements) in the sample. The EDXRF process may be referred to as a fluorescence response-based analytical process.

EDXRF may be considered a non-destructive analytical technique used to determine the elemental composition of materials. EDXRF analyzers determine the elemental composition of a sample by measuring the fluorescent (or secondary detectable energy) X-ray emitted from a sample when it is excited by a primary X-ray source. EDXRF is designed to analyze groups of elements simultaneously to determine those elements presence in the sample and their relative concentrations-in other words, the elemental composition of the sample. Each of the elements present in a sample produces a unique set of characteristic X-rays that is a “fingerprint” for that specific element. X-rays have a very short wavelength, which corresponds to very high energy. All atoms have several electron orbitals (K shell, L shell, M shell, for example). When X-ray energy causes electrons to transfer in and out of these shell levels, X-ray fluorescence peaks with varying intensities are created and will be present in the spectrum. The peak energy identifies the element, and the peak height or intensity is indicative of its concentration.

The term “XRD” may refer to X-ray diffraction, which is a technique for analyzing the atomic or molecular structure of materials. It is non-destructive, and works most effectively with materials that are wholly, or part, crystalline. The technique is often known as x-ray powder diffraction because the material being analyzed typically is a finely ground down to a uniform state. Diffraction is when light bends slightly as it passes around the edge of an object or encounters an obstacle or aperture. The degree to which it occurs depends on the relative size of a wavelength compared to the dimensions of the obstacle or aperture it encounters.

All diffraction methods start with the emission of x-rays from a cathode tube or rotating target, which is then focused at a sample. By collecting the diffracted x-rays, the sample's structure can be analyzed. This is possible because each mineral has a unique set of d-spacings. D-spacings are the distances between planes of atoms, which cause diffraction peaks.

The term “flow mapping” as used herein may refer to the process of analyzing and measuring the movement of fluids within and between various wells and from each frac stage and the surrounding reservoir. A resultant flow mapping parameter may help in understanding the flow patterns of hydrocarbons and water, often essential for optimizing production, refining the well and frac designs, and improving recovery techniques. Interpreting the flow patterns based on solid tracers can identify issues such as reservoir heterogeneity, fluid movement behavior, and potential flow blockages. Flow mapping may help in making informed decisions about drilling, production strategies, and enhanced recovery methods. Monitoring of flow data over time using solid tracers can detect changes in well and stage-specific flow performance, which can then drive future re-frac schedules or intervention strategies. Overall, flow mapping is a crucial tool in optimizing oil and gas extraction processes and maximizing resource recovery.

The term “non-magnetic” as used herein may refer to physical property understood by one of ordinary skill in the art as not having any magnetic behavior or properties. Therefore, embodiments of the disclosure may be completely void of using magnets or anything having magnetic features.

The term “completion quality” (CQ) as used herein may refer to an engineering evaluation of the factors that influence the effectiveness of hydraulic fracture treatments. This includes the ability to initiate and establish an induced fracture network, the extent of reservoir contact made by the newly created fractures, the level of connectivity to the wellbore, the existing natural fracture system, and the stimulated reservoir's capacity to deliver gas or oil to the well.

The term “reservoir quality” (RQ) as used herein may refer to a geologic, engineering, and economic assessment of a wellbore, its reserves, and its producibility. RQ may include factors such as the volume of oil or gas in place, total organic content (TOC), thermal maturity, effective porosity, fluid saturations (oil, gas, and water), reservoir thickness, and intrinsic permeability.

Embodiments herein may used or associated with a method of using tracers in a key role for delivery of useful data that lead to a comprehensive understanding of the reservoir/formation, geological faults, and optimize stimulation efforts at a stage level. Additional multi-well studies may in turn lead to the development of a full understanding of inter-well communication, fracture-driven interactions flow characterization, and optimizing well spacing.

According to embodiments, tracers may be added to each stage during completion for flow profile mapping and fault evaluation. In addition, a sampling campaign of adjacent wells may be initiated to identify potential communication between offset and traced wells. The data integration of the tracer data with offset wells pressure monitoring may improve the target reservoir development approach and provide a live performance flow profile for the entire development. Tracer data may be used to optimize completion designs, stage and well placement strategies, and significantly enhance reservoir understanding and geological fault impacts on stage-level production.

Embodiments herein may be used for characterization of formation evaluation and hydraulic fracture treatment evaluation, which may include non-destructive tracer usage that does not interfere with wellbore operation.

Enhanced by advancements in nanoparticle science and advancement, these robust, non-radioactive, and non-hazardous tracers may be engineered with subatomic spectroscopic measurement techniques. Embodiments thus provide for effective map well production distribution, assess the performance of each stage, monitor cross-well interference, and evaluate reservoir conditions.

Any tracer of embodiments herein may be easily deployable, distinctly tagged and non-intrusive tracers, which may be able to maintain stability even at extremely high temperatures.

In contrast to standard chemically based soluble tracers, tracer embodiments herein may remain intact within the fracture network, allowing for continuous on-site monitoring of the flow profile, which may occur for an extended duration of time, such as for up to 10 months, and sometimes longer.

Tracers of the present disclosure may be able to convey much more information about fractures in the reservoir, from the flow characteristics to the fracture's conductivity. Easily deployed, clearly tagged, safe, non-radioactive tracers may maintain stability at excessive reservoir temperatures (e.g., such as up to 2,000° F.), have a light specific gravity (e.g., 0.9-1.17 g/cm), and/or withstand the high closure stress (e.g., such as up to 10,000 psi).

This may compare very favorable as compared to limitations of standard chemical tracers. Tracer embodiments herein may remain intact within the fracture networks, allowing continuous on-site monitoring of the flow profile.

Embodiments herein may include the step of disposing one or more tracers into respective one or more stages of a wellbore. A formation or remnant fluid may be produced from the wellbore, the formation fluid may include any of the tracers. A sample of the formation fluid may be taken and tested. The method may include testing the sample in accordance with embodiments herein to obtain or provide a set of data. The set of data may be assessed or converted in a manner to provide an indication of wellbore performance. Wellbore performance may pertain to production contribution, production signature compared to average, long term short term, reservoir quality and completion quality. Conversion of the state of data may be via computer software. The indication may be a visual indicator, such as a graph depicting reservoir quality and completion quality for one or more stages of the wellbore.

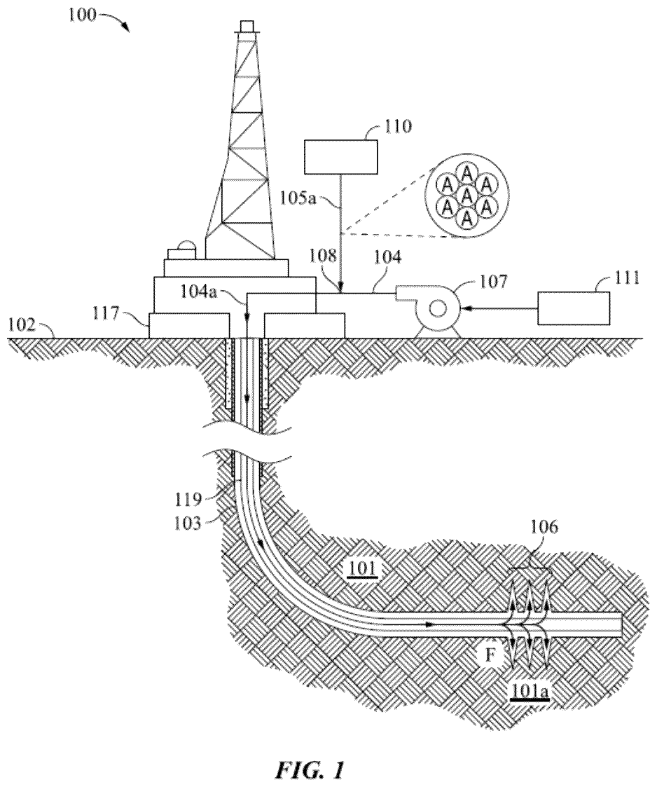

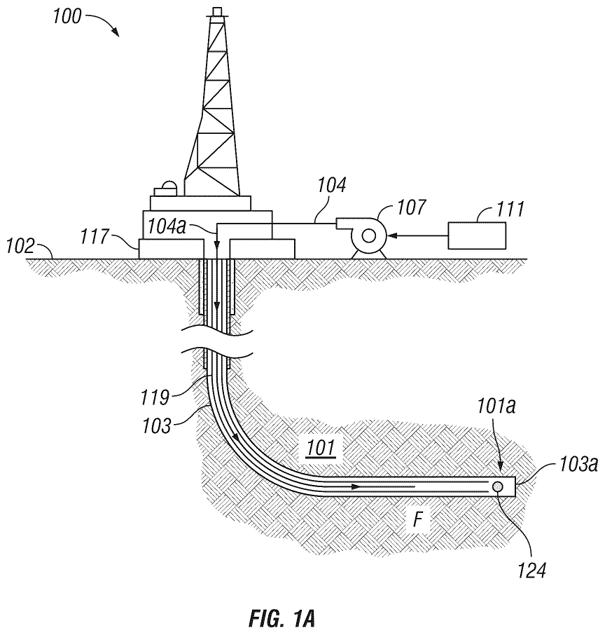

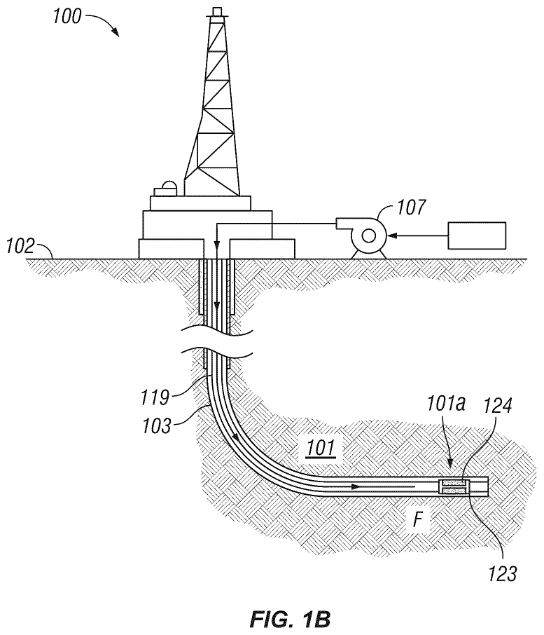

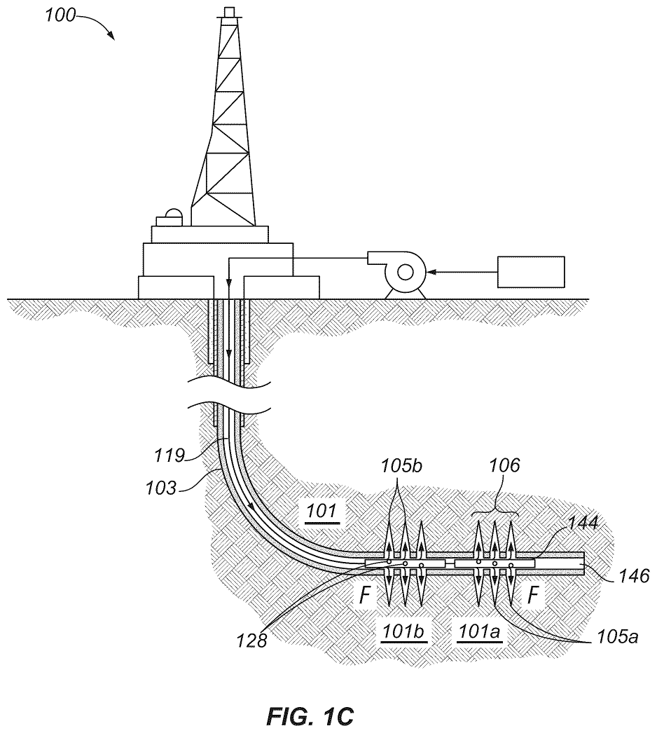

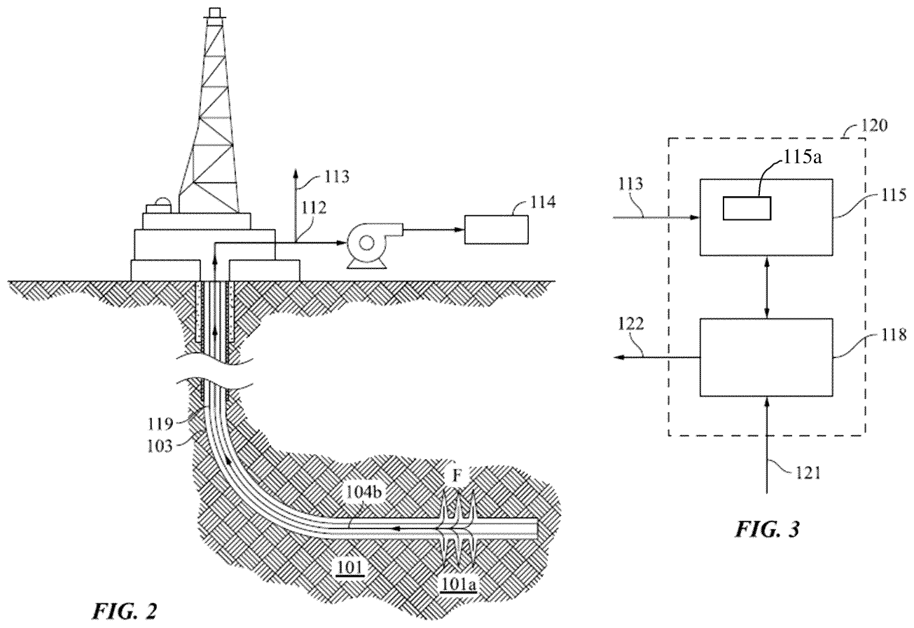

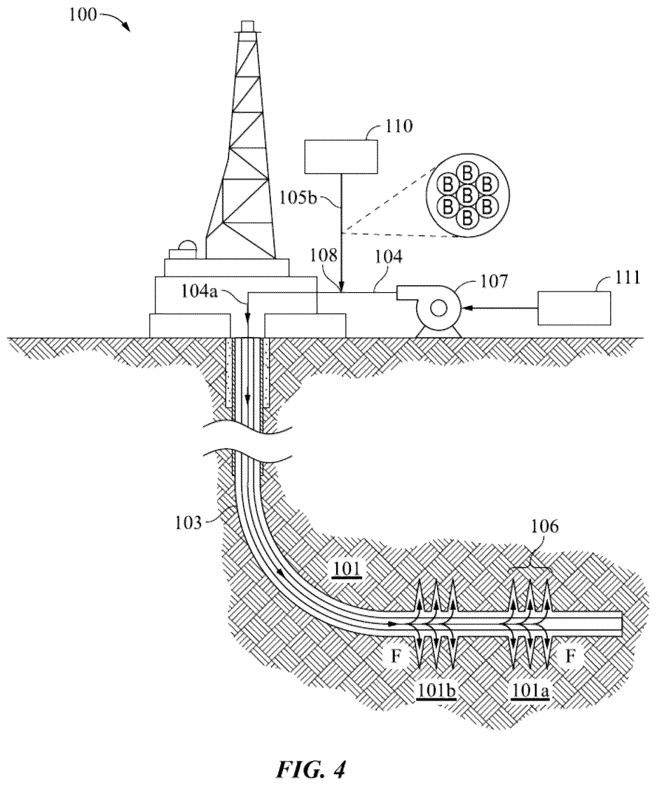

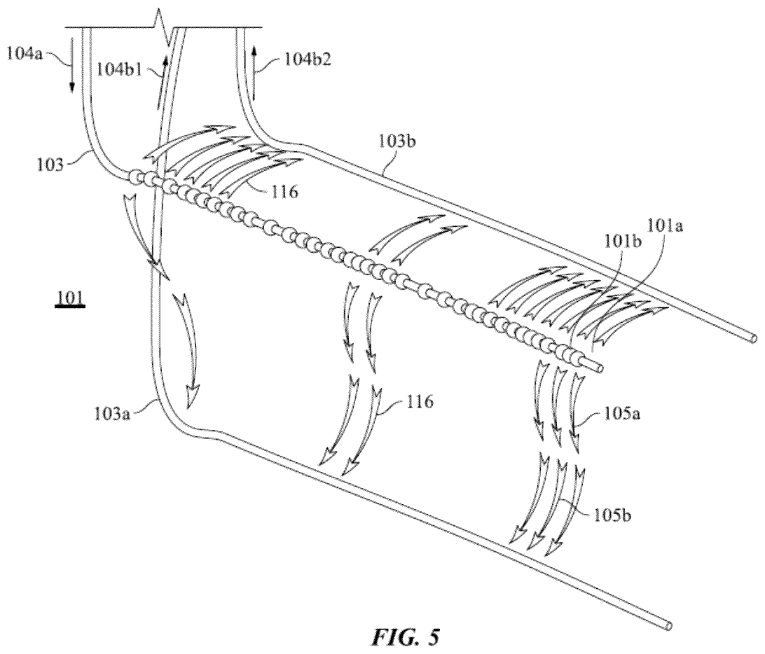

Referring now to FIG. 1, FIG. 2, FIG. 3, FIG. 4, and FIG. 5 together, a side view of a system (and related method) for using a tracer in a wellbore; a side view of the system of FIG. 1 where a remnant fluid with the tracer is produced from the wellbore; a simplified block diagram of an analytical testing unit used to test a sample having a tracer; a side view of the system of FIG. 1 for using a second tracer in a wellbore; and a side view of a system for using a tracer in a formation having multiple wellbores, respectively, according to embodiments disclosed herein, are shown.

System 100 may include one or more components (or subcomponents) coupled with new, existing, or retrofitted equipment. System 100 may include one or more units that are skid mounted or may be a collection of skid units, and the system 100 may be suitable for onshore and offshore environments.

The system 100 may have various valves, flanges, pipes, pumps, utilities, monitors, sensors, controllers, flow meters, safety devices, etc., for accommodating sufficient universal coupling between system components and any applicable feedline/feed source of a material to be processed, any resultant product material to be discharged or transferred therefrom, and anything in between.

FIG. 1 is meant to show in a simplistic manner embodiments herein, and may not be to scale. The system 100 may include a subterranean or earthen formation 101 having a wellbore 103 drilled or otherwise formed therein. The formation 101 may be a type of conventional or unconventional reservoir. The formation 101 may contain hydrocarbonaceous fluids, such as oil, natural gas, and/or other materials, generally designated as F. The formation 101 may include porous and permeable rock containing liquid and/or gaseous hydrocarbons. The formation may include a conventional reservoir, an unconventional reservoir, a tight gas reservoir, and/or other types of reservoirs. Moreover, the illustration of a mover (pump) 107 is not meant to infer other equipment is not present, of which one of ordinary skill in the art is well versed.

The system 100 may include one or more additional wellbores, production wells, etc. The example wellbore 103 shown in FIG. 1 illustrates the wellbore 103 may have at least a partial horizontal trajectory. However, any wellbore of the system 100 may include any combination of horizontal, vertical, slant, curved, directional-drilled, and/or other well geometries.

The wellbore 103 may be open, closed, cased, uncased, etc. Although not shown in detail here, the wellbore 103 may have a tubestring 119 disposed therein, such as for deploying tools or fluids into the wellbore 103. In other aspects, the tubestring 119 may be a production tubing, whereby formation and wellbore fluids may be readily transported to a surface or surface facility 102.

The formation 101 may include a target formation 101a, which may be believed to be a hydrocarbon-rich area of the formation 101. The target formation 101a may be a stage or zone, which may be part of or associated with a fracing operation. Just the same, the target formation 101a may just be part of the formation 101 without the need for enhanced oil recovery (EOR) or other type of treatment.

In the event of EOR, and although not limited to any particular type of EOR/IOR or comparable operation, in multistage fracturing, the wellbore 103 may require the stimulation and production of one or more zones of a formation. Conventionally, a liner, casing, or other type of tubestring 119 may be used downhole, in which the tubestring 119 includes one or more downhole frac valves (any may further include, but not be limited to, ported sleeves or collars) at spaced intervals along the wellbore. Just the same, the target formation 101a may be fractured with a plug-and-perf operation.

It may be the case that the target formation 101a has perforations 106. The perforations may result from a fracing operation or may naturally exist. The perforations 106 shown here are exaggerated in scale for ease of understanding to the reader, but may in reality be small in scale.

In the event of tight formation characteristics, such as in the case of an unconventional reservoir, the target formation 101a may have an average permeability of about 0.1 nanodarcy to about 1000 nanodarcy. By way of comparison, the target formation 101a may be disposed in a conventional reservoir, and thus may have an average permeability in a range of about 0.1 millidarcy to about 1 darcy (or more).

The formation 101 might have other geologic characteristics, including hot formation temperatures. For example, the target formation 101a may have an average formation temperature T of about 450° F. In embodiments, the average formation temperature T may be in a range of about 100° F. to about 800° F. The formation temperature T may have a relationship to the depth, geological environment, and tightness of the formation 101.

Diagnostic information about the performance of the wellbore 103 may be determined by utilizing a first tracer (additive) 105a. The first tracer 105a (or other tracers described herein) may be of a suitable material for use with any type of formation 101. Just the same, the first tracer 105a may have a (predetermined) first composition A, which results in characteristics (or traits) suitable for use in the event the formation 101 has conditions normally undesirable for the use of tracers, namely, liquid tracers.

As a first characteristic, the first tracer 105a may be a solid tracer in the form of a powder. The use of powder form makes the first tracer 105a attractive for use in high temperature conditions. The first tracer 105a may comprise powder (nano)particles. In embodiments, the particles of the first tracer 105a may have an average particle diameter of about 0.1 μm to about 10 μm. The first tracer 105a may have a first tracer specific gravity. In embodiments, the first tracer 105a may have an average bulk specific gravity of about 0.6 to about 1.2. The first tracer 105a may have a physical property of being non-magnetic, and thus non-responsive to any kind of magnet device. The first tracer 105a may be inert, and as such may be non-destructive to the wellbore.

The first tracer 105a may be transferred (e.g., a blower, pump, gravity feed, etc.) from a tracer feed source 110 (such as a hopper or the like), and mixed with a carrier fluid 104. The carrier fluid 104 may be or include water. Other materials may be mixed with the carrier fluid, such as sand, proppant, etc. The carrier fluid 104 may be transferred toward mixer or mixing point 108 from a carrier fluid source 111. For example, a pump 107 may be used to pump the carrier fluid 104 from the source 111 toward a wellhead (injection point) 117, and through the tubestring 119.

The mixer 108 may be any device suitable to form an injection or utility fluid mixture 104a. The first tracer 105a may be completely miscible with the carrier fluid 104. The first tracer 105a may be inert in the respect that there is no effect by the first tracer 105a on the carrier fluid 104 and/or the formation 101 (or target formation 101a) and/or vice versa.

Suitable equipment such as the pump 107 may be used for the transfer and disposing of the utility mixture 104a into the wellbore 103. As may be seen, the utility mixture 104a may be disposed into the same wellbore 103 from which the tracer 105a may be later produced. Sufficient pressure and flowrate may be selected and used in order to adequately provide the utility mixture 104a to the target formation 101a. The first tracer 105a may exit the tubestring 119, and eventually come into contact with the target formation 101a and/or the perforations 106. The tracer 105a may be provided to the target formation 101a through standard flow configurations, such as ports/sleeves within the tubestring 119, or out of a toe, and vice versa for production to the surface 102.

The tracer 105a (or at least a portion thereof) may have an average residence time in the target formation 101a and/or the perforations 106. In the event the system 100 uses a fracing operation for a stage or a zone, the first tracer 105a may be selected for its particular uniqueness, and thus preferably has a different tracer characteristic (fingerprint) from other tracers used in the fracing operation so that fluid returned from each particular stage may be identified. The tracer characteristic may be the chemical identity of the tracer used, such as composition or specific gravity. The tracer characteristic may be distinguishable from the tracer characteristic(s) of any other tracers used.

Embodiments herein are not limited to pumping a tracer fluid into the wellbore. For example, referring briefly to FIG. 1A, a side view of a system for using a deployment device for the transfer of a tracer into a wellbore, according to embodiments disclosed herein, is shown.

FIG. 1A shows the first tracer 105a may be disposed into a first deployment device 124. Although not meant to be limited, the first deployment device 124 may be spherical in nature. The deployment device 124 may have an outer diameter. The outer diameter may be in the range of about 2 inches to about 5 inches.

The deployment device 124 may be sent or disposed into the wellbore 103 via carrier fluid 104. For example, the pump 107 may be used to pump the carrier fluid 104 from the source 111 toward a wellhead (injection point) 117, and through the tubestring 119.

Sufficient pressure and flowrate may be selected and used in order to adequately provide the deployment device 124 to the target formation 101a. After an amount of time (which may be predetermined or otherwise known), the deployment device 124 may dissolve sufficiently enough that the first tracer 105a may begin to disperse in or at the target formation 101a. The amount of time for dispersion to begin may be about 24 hours to about 60 hours. In embodiments, the amount of time may be about 2 days.

The first tracer 105a may be completely miscible with the wellbore fluids. The first tracer 105a may be inert in the respect that there is no effect by the first tracer 105a on the carrier fluid 104 and/or the formation 101 (or target formation 101a) and/or vice versa.

As another example, referring briefly to FIG. 1B, a side view of a system for using a downhole tool for the transfer of a tracer into a wellbore, according to embodiments disclosed herein, is shown.

The first tracer 105a may be associated with or disposed into/onto a downhole tool 123. The downhole tool 123 may be coupled with or part of the tubestring 119. The downhole tool 123 may be any kind of tool suitable for use in the wellbore 103, such as a frac plug (or other desired completion tool), a drill bit (or part thereof), a (sand) screen, and so forth. The downhole tool 123 may be, or include a component part thereof, configured to hold, convey, and subsequently deploy, the first tracer 105a.

As shown here, the downhole tool 123 may have a first deployment device 124. Although not meant to be limited, the first deployment device 124 may be cylindrical or elongated in nature.

The downhole tool 123 may be a completion tool or the like. The downhole tool may be configured for a deployment device 124 therein. In aspects, there may be one or more devices 124. The downhole tool 103 (or deployment device) may have any tracer (e.g., 105a) of the like described for embodiments herein associated with, or otherwise disposed therein. In aspects, the deployment device 124 may have the tracer disposed within, such that the tracer may be conveyed into the wellbore 103 for downhole deployment (release, etc.) (in contrast to surface deployment).

To prevent the tracer from inadvertent deployment or contact with the target formation (or associated wellbore fluid(s) F), the downhole tool 123 and/or the deployment device 124 may include a dissolvable material. In this respect, the tracer 105a may be prevented from deployment until the dissolvable material has dissolved in a sufficient manner. Once released, the tracer 105a may mingle, disperse, interact, etc. with the target formation 101 and/or fluid(s) F. The fluid For a sample thereof (which may have at least some of the tracer 105a and at least home hydrocarbon component) may be returned to the surface, and subsequently tested.

FIG. 1B shows the first tracer 105a for illustrative purpose, although the tracer 105a may not be visible unless or until the deployment device 124 (sufficiently) dissolves. On the other hand, there may be one or more embodiments where the first tracer 105a may be visible (or more exposed) or part of a surface component or coating.

The amount of time for dispersion to begin may be about 24 hours to about 60 hours, but could also be longer. In embodiments, the amount of time may be about 2 days. In other embodiments, some applications may require days, weeks or even months before the tracer be released. How long it takes for the first tracer 105a to be released may be designed. The release time may also be based on the amount and/o composition of dissolvable material used, as well as whatever monitoring objective may be desired or used.

As yet another example, referring briefly to FIG. 1C, a side view of a system for using a perforator device for the transfer of a tracer into a wellbore, according to embodiments disclosed herein, is shown.

The wellbore 103 may have a casing (casing string, etc.) 146 disposed therein, which may be held in place or otherwise secured via cement. There may be a workstring 119 configured with one or more perforator devices 144. When it is desired to perforate the formation 101 (or, e.g., a target formation thereof), the workstring 119 may be lowered through casing 146 until the devices 144 are properly positioned relative to the formation 101/target formation. There may be a plurality of target formations (or stages) 101a, 101b, etc.

One or more shaped charges 128 of respective device(s) 144 may be sequentially fired, either in an uphole to downhole or a downhole to uphole direction, or as desired. Upon detonation, the shaped charges 128 may form explosive material jets sufficient create perforations 106 extending outwardly through casing 146, cement and into the formation 101, thereby allow formation communication between the formation 101 and the wellbore 013.

Each of the shaped charges 128 may have a tracer associated therewith. As such, each jet may convey the tracer into the formation 101. Just the same, each shaped charge 128 may have multiple different types of tracers associated therewith. For example, the shaped charge may have a first tracer, a second tracer, a third tracer, etc. associated therewith.

Returning now to FIG. 1, FIG. 2, FIG. 3, FIG. 4, and FIG. 5 together, FIGS. 2 and 3 illustrate whereby the first tracer 105a may be brought back to the surface 102 (although not limited, this may be from the same wellbore 103 it was introduced) for testing. For example, after the predetermined time period, a remnant fluid 104b may be produced. The remnant fluid 104b may include, at least partially, (some of) the first tracer 105a, the carrier fluid, and formation fluids F. A sample of the remnant fluid 104b may be produced on a desired frequency, such as daily. The sampling can occur during the desired frequency over a predetermined timeframe, which may be days or months (e.g., 6 months).

Once the remnant fluid 104b is produced from the wellbore 103, a sample 113 may be taken or extracted from sample point 112. The rest of the remnant fluid 104b may be transferred to a desired destination 114, which may be a tank, a pond, another well, or other suitable storage.

The sample 113 may now be tested via test unit 120. Prior to or as part of testing, the sample 113 may be filtered, resulting in a filtered sample 113. Filtration may occur, for example, by membrane filtration. Filtration may occur at or with test unit 120.

The test unit 120 may include analysis equipment 115, which may be in operable communication with computing system 118. The test unit 120 (and/or analysis equipment) may include filtration equipment 115a. The computing system 118 may be configured for use in using analytical data associated with use of the test equipment 115. The test equipment 115 may provide a fluorescence response-based process, such as EDXRF and XRD.

The computing system 118 may be useful to further analyze data and other information in order to provide an indication related to performance of the wellbore 103. This may pertain to, for example, the time the tracer was detected, the location where the tracer was used, the type and composition of the tracer detected, the amount or concentration of tracer detected, and/or other measurements provided by the equipment 115 and the system 118.

The computing system 118 may have artificial intelligence (A.I.) based diagnostics. The computing system 118 may access input data 121, which may be related to other aspects of the formation 101, such as geological information, fractures, and the like. The computing system 118 may include programs, scripts, and/or other types of computer instructions that generate output data 122 (i.e., set of data, data set, etc.), which may be based on the input data 121. The output data 122 may include descriptions of fluid flow patterns in the formation 101, which may identify paths of fluid flow in the wellbore 101, wellbore breaches or cross-communication (such as to a proximate offset well), fracture locations, fluid flow rates, and/or other information. For example, the output data 122 may be used for flow mapping the wellbore 103.

The output data 122 may be used to provide an indication of reservoir quality, completion quality, and combinations thereof.

FIG. 4 illustrates an analogous manner of disposing a second tracer 105b into the wellbore 103, which may occur via any suitable manner, such as examples provided herein. The second tracer 105b may be like that of the first tracer 105b, and thus have similar composition and characteristics; however, the second tracer may have a second composition B different from that of the first composition A. The use of a different composition B provides a unique identifier and fingerprint as compared to that of the composition A.

The second composition B may be different from the first composition A, yet the second tracer 105b may have characteristics similar to that of the first tracer 105a. For example, the second tracer 105b may be an inert solid (in powder form) having a respective average particle diameter of about 0.1 μm to about 10 μm. The second tracer 105b may have a respective average bulk specific gravity of about 0.6 to about 1.2.

The second tracer 105b may be transferred from the tracer feed source 110, and mixed with the carrier fluid 104 at mixer or mixing point 108 to form the utility mixture 104a. The utility mixture in this instance may thus include the carrier fluid 104 and the second tracer 105b. Sufficient pressure and flowrate may be selected and used in order to adequately provide the utility mixture 104a to a new or second target formation 101b. The second target formation 101b may be associated with a stage or zone of a frac operation. Just the same the second target formation 101b may just be part of the formation 101. The second target formation 101b may have its own respective perforations 106.

As before with the first tracer 105a, after the predetermined time period, a remnant fluid 104b may be produced. The remnant fluid 104b may include, at least partially, (some of) the first tracer 105a, the second tracer 105b, the carrier fluid, and formation fluids F.

Once the remnant fluid 104b is produced from the wellbore 103, a sample 113 may be taken or extracted from sample point 112.

The system 100 may be modified or adjusted based on the detection of tracers released from the formation 101. For example, well system tools, and/or other subsystems may be installed, adjusted, activated, terminated, or otherwise modified based on the information provided by the tracers. Additional fractures can be formed in the formation 101, and/or other modifications can be made based on information provided by the tracers. In some embodiments, modifications of the system 100 may be selected and/or parameterized to improve production from the formation 101. For example, the modifications may improve the sweep efficiency. Modifications of well system 100 may be selected and/or parameterized by the computing system based on data analysis performed by the computing system.

Briefly, FIG. 5 illustrates an embodiment where a utility fluid 104a may disposed into a wellbore 103 in a suitable manner to provide a first tracer 105a to a first target formation 101a, and subsequently a second tracer 105b to a second target formation 101b. Other tracers may be added for other areas of the formation 101, such as other stages. The tracers 105a, 105b may be like that as described herein.

As shown here, there may be a plurality of wellbores, such as first offset wellbore 103a and second offset wellbore 103b. In some embodiments, it may be desirous to establish far-field analysis or otherwise detect cross-communication 116 or breakthrough between the wellbores 103, 103a, 103b. As shown here, the first tracer 105a may pass from the wellbore 103 into any of the offset wellbores via flowpath 116. Similarlily, the second tracer 105b may pass from the wellbore 103 to any of the offset wellbores via flowpath 116. Other tracers may be used and passed, as well.

Remnant fluids 104b1, 104b2 may be produced from the respective wellbores and have samples taken and tested in accordance with embodiments herein.

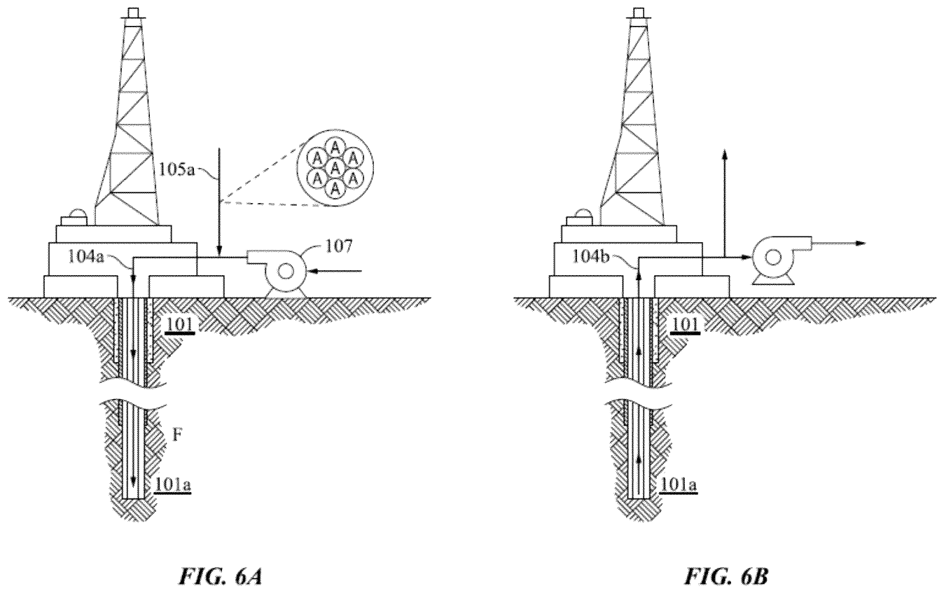

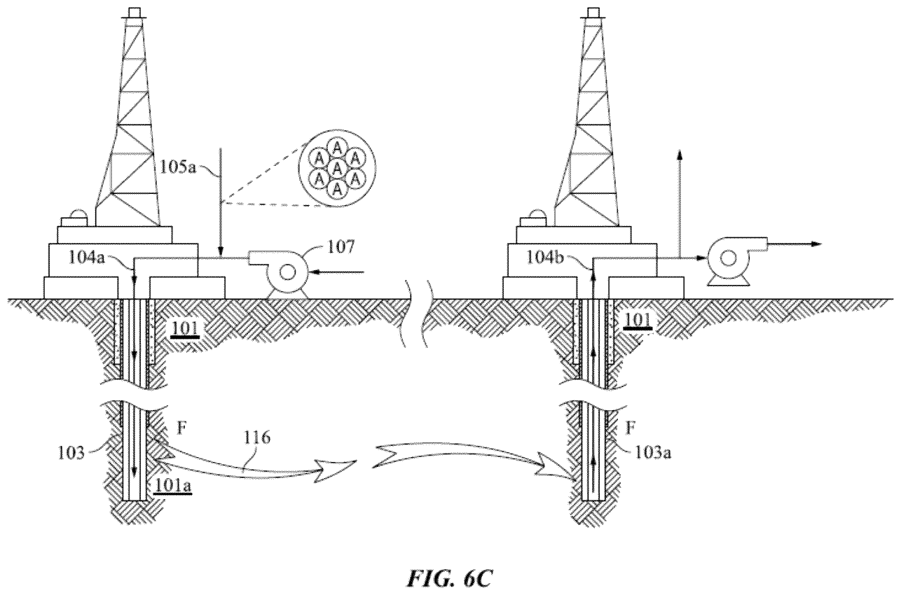

Briefly, FIG. 6C illustrates a side view of a system for using a tracer in a geothermal well, and a side view where a remnant fluid with the tracer is produced from another nearby well. For example, cross-communication 116 may exist between the wellbore 103 and the nearby wellbore 103a. As such, the remnant fluid 104b may be produced from the nearby wellbore 103a.

### Example 1

Embodiments herein provide for a method of subsurface flow mapping using ultrahigh resolution nanoparticle tracer technology. Methods of the disclosure may provide for a tracer portfolio that integrates advanced computational methods using Artificial Intelligence (A.I.). Such use may provide accurate, actionable, near real-time performance-flow-profile data. This may allow oil and gas operators to: optimize completion strategies; achieve the best production per foot; reduce completion and fracturing cost; and/or reduce environmental footprint.

Tracer technology described herein may be based on proprietary inert, non-magnetic submicron particles and other environmentally friendly and cost-effective additives that are used to manufacture the right composition of each tracer. This tracer technology may utilize special inert particles fingerprinting with certain atoms as special indicators that enhance the properties of each tracer. These may then detected at the sub-atomic structure level using robust capabilities of EDXRF-type spectroscopy measurements, and therefore ensuring superior accuracy for each tracer's detection and characterization from different subsurface environments.

The tracer additives may be injected at the parts per million (PPM) concentrations via mixer or blender equipment, and pumped (injected) downhole into well using high-pressure pumps for any given fracturing stage. The mixer equipment may be designed to integrate a slurry of water, sand, dry chemicals, and liquid chemicals to provide the desired fracing components and fluid rheology for any target formation.

After a completion process is completed and the well flown back, the deployed tracers are then recovered with production flowback or produced fluids from treatment or/and adjacent wells.

During the back flowing of the well, reservoir oil/gas samples are taken on a regular basis, such as for the first 10 to 40 days. The number of days may as desired, such as up to 180 days. A small amount of the sample is analyzed using appropriate methods to detect the presence and concentration of tracer compound. Samples from traced and/or offset wells may be collected on a predetermined basis (such as daily) from production flowback at the wellhead or other suitable sample point. The sample may then be tested via a fluorescence response-based process, such as EDXRF and XRD. Such analytical techniques may be used to determine the elemental composition and crystallinity of the samples.

EDXRF is designed to analyze groups of elements simultaneously to determine those elements presence in the sample and their relative concentrations—in other words, the elemental composition of the sample. Each of the elements present in a sample produces a unique set of characteristic X-rays that is a “fingerprint” for that specific element. X-rays have a very short wavelength, which corresponds to very high energy.

Due to sub-atomic accuracy of both detection methods, it is possible to precisely determine the elemental composition, crystallographic structure, and the various combinations of hyperfine interactions in the samples, which enables very accurate identification of the tracer additives on the sub-atomic or quantum level.

Laboratory analysis that may include or incorporate advanced computational methods and proprietary diagnostics capabilities for each stage or target formation provides accurate, calibrated, actionable and cost-effective production diagnostics results. This enables operators to reduce operational cost and increase the production in oil and gas wells.

Embodiments herein may produce and achieve an extensive and long-term dataset from tracer additives during production flow profile analysis at each target formation. This information may be used together with advanced computational methods using Artificial Intelligence (A.I.) coupled with artificial neural network may provide precise completion optimization workflows for oil and gas wells.

Embodiments herein pertain to a method(s) of using a tracer in a wellbore. The method may include one or more steps, which may vary in sequence and scope. The method may include forming a utility fluid mixture comprising the tracer. Forming may occur from inline mixing of the like. The utility fluid may then be disposed or otherwise transferred into the wellbore.

The utility fluid may be disposed at a sufficient flow rate and pressure so that the utility fluid comes into contact with a target formation in communication with the wellbore. A such, the flow rate and pressure may be adequate to transfer the utility fluid through a tubestring, into the wellbore, and then into contact with the target formation (such as through sleeve ports, a toe sleeve, or the like).

Upon contacting the utility fluid with the target formation for an amount of time (which may be predetermined or as otherwise desired), the method may include returning (producing, etc.) a remnant fluid to a surface. One of skill would appreciate the surface refers to above-ground production equipment, facilities, and so forth, being common in fracing operations.

The method may include taking a sample of the remnant fluid, and then testing the sample in order to analyze the remnant fluid in order to provide a set of fluid data. The method may include filtering the sample or remnant fluid to provide a filtered version of the same.

The method may include integrating (or otherwise analyzing, comparing, etc.) the set of fluid data with other wellbore data in order to determine a parameter associated with performance of the wellbore.

The method may utilize the tracer having a first tracer composition. The tracer may be in powder (i.e., solid) form having an average particle diameter of at least 0.1 μm to no more than 10 μm (or any range therebetween). The tracer may have an average bulk specific gravity. For example, the average bulk specific gravity may be in a range of at least 0.6 to no more than 1.2.

The wellbore may be associated with extreme conditions, such as formation temperature of at least 1000° F. to no more than 2000° F. The formation (or the target formation) may have an average permeability of 0.1 nanodarcy to 1000 nanodarcy. In aspects, the target formation may be part of a geothermal well. In this respect, the remnant fluid may be used in an energy generation process.

The method may include additional steps, such as disposing a second tracer into the wellbore. The disposing step may be done in such a manner that the second tracer may come into contact with one or more of the target formation, another target formation proximate to the wellbore, or combinations thereof.

The second tracer may have a different composition from the first chemical tracer, but otherwise may also be in powder form, may have an average particle diameter of at least 0.1 μm to no more than 10 μm, and may have average bulk specific gravity of at least 0.6 to no more than 1.2.

The target formation may be associated with a frac stage, wherein the wellbore is associated with a formation temperature of at least 450° F. to no more than 2000° F., and wherein the target formation has an average permeability of 0.1 nanodarcy to 1000 nanodarcy. The testing the sample step may include using a fluorescence response-based analysis. In aspects, the fluorescence response-based analysis comprises EDXRF. In aspects, the fluorescence response-based analysis comprises XDR.

Any tracer of the present disclosure may be entirely non-magnetic.

Any sample or remnant fluid of the present disclosure may be filtered prior to or as part of testing and analysis.

### Example 2—Tracer Use and Methodology

An example dispersion and usage of tracers occurred. For each stage of the wellbore, a specific tracer was designed and manufactured (e.g., unique ultra-high-resolution nanoparticle). In total, 63 unique tracers were produced for the 63 stages of this well. Each stage utilized 7 pounds of nanoparticle tracer, injected at a concentration of 15.5 parts per million (ppm). The production of each tracer adhered to rigorous manufacturing, quality control, and testing protocols. Prior to deployment, each tracer underwent stringent lab sub-atomic testing and validation processes to ensure optimal downhole performance and flow efficiency. Additionally, all tracers were pre-mixed in a specialized liquid to ensure occupational safety for the tracer crew and to ensure accurate injection during each stage. The nanoparticle tracers are non-magnetic, solid, inert, non-radioactive, and non-hazardous materials, hence not interacting with frac fluids.

Prior to the project's start, the tracer equipment rig-up and deployment were thoroughly discussed with the frac crew and operator's field superintendents. The frac pumping company facilitated access to the blender and provided a radio for communication with the frac crew and the data/control van. All tracers were pre-mixed in a specialized suspension liquid and delivered onsite in 1-gallon containers (jars). During pumping, the nanoparticle tracers were added to the frac blender tub to ensure excellent mixing and dispersion. The nanoparticle tracer injection commenced after the stage pad phase when frac sand was entering the frac blender and was halted just before the stage flush phase. This approach enabled uniform nanoparticle tracer placement within the hydraulic fracturing (prop area inside the frac) for each stage and prevented cross-stage tracer contamination.

Frac operations began and completed within approximately one month. The injection and deployment of all tracers proceeded as scheduled, without any interruptions or injection issues. Data from the nanoparticle tracer operations were recorded and streamed live to support personnel and office experts. Upon completion of the wellbore, a tracer deployment report was submitted, including the injection log and the start and stop times for nanoparticle tracer injection at each stage.

Collected samples were delivered to the sub-atomic analytical laboratory for comprehensive tracer analysis. Using an extraction process, particles were extracted from the oil and water phases of each sample. These particles were then analyzed using advanced, established techniques: energy dispersive X-ray fluorescence (EDXRF) and X-ray diffraction (XRD).

EDXRF is a non-destructive analytical technique used to determine the elemental composition of materials. EDXRF analyzers determine the elemental composition of a sample by measuring the fluorescent (or secondary) X-ray emitted from a sample when it is excited by a primary X-ray source. X-rays have a very short wavelength, which corresponds to very high excitation energy. When X-ray energy causes electrons to transfer in and out of these shell levels, X-ray fluorescence peaks with varying intensities are created and will be present in the final measurement spectrum. The peak energy identifies the element, and the peak height or intensity is indicative of its concentration for comprehensive qualification and quantification in elemental analysis. EDXRF technology is a non-distractive, portable, and comprehensive way to screen all kinds of particles for quick identification and quantification of elements.

XRD is a non-destructive technique that provides detailed information about the crystallographic structure, composition, and physical properties of nano particles. XRD is a rapid analytical technique primarily used for phase identification and the crystallinity of a sample. XRD is widely used for the identification of unknown crystalline materials (e.g., minerals, metals, oxides, and inorganic compounds). Many industries rely on proven XRD results to characterize materials, from basic product research all the way to quality control and quality assurance. A sample is placed in the path of an X-ray beam. X-rays diffract through the crystal and into a detector. The beam and detector are rotated through a range of angles. The angles at which the crystals diffract the beam into the detector correspond to the planes of the crystals. Each crystal has a characteristic pattern of diffraction angles and the corresponding intensity of the diffracted beam. Once the beam is separated, the scatter, also called a diffraction pattern, indicates the sample's crystalline structure.

Thanks to the sub-atomic precision of both detection methods, it is possible to accurately ascertain the elemental composition, crystallographic structure, and various interaction combinations as distinct signatures within the samples. This facilitates highly accurate identification of tracers at the sub-atomic or quantum level. The results from tracer detection generated a comprehensive long-term dataset regarding nanoparticle tracer recovery in both oil and water phases, along with flow profile information for each stage. When combined with pressure diagnostics and geomechanics integration, this data enabled the most precise completion optimization workflows for the wellbore.

A table of data can be used to illustrate the tracer recovery for each of the 63 stages. An upper plot can show the recovery of tracers in the oil phase (or oil tracers), averaged across the first 5 samples and the last 5 samples. A lower plot can display the recovery of tracers in the water phase (or water tracers), also averaged for the first 5 samples and the last 5 samples. All tracers were detected in parts per billion (ppb) for each oil and water sample, reflecting the flow profile for each stage.

By weighing the production rate from each stage by its corresponding tracer recovery concentration, the contributions to the stage-level production rate can be derived from the nanoparticle tracer recovery data

By analyzing the well-established production rate over time and the stage-by-stage contributions to production, valuable insights into stage-level production can be gained. For instance, Table 3 below displays the production data for each stage over 29 days and 167 days, alongside the average production for each stage based on the assumption that all stages perform equally. As indicated in the Table, certain stages exceed the average production, while others fall below it. The variations in stage-level production performance may be attributed to differences in completion quality (CQ) and reservoir quality (RQ).

Monitoring and measuring stage-level production can provide valuable insights into the completion and reservoir qualities of each stage along the entire horizontal lateral. For this analysis, the following assumptions were made:

Short-term production (e.g., 30-day or 29-day, in this case) is utilized as a proxy for completion quality (CQ), serving as a rough indicator of the conductivity of the proppant-packed fracture and its connection to the wellbore.

Long-term production (e.g., 180-day or 167-day, in this case) is employed as a proxy for reservoir quality (RQ).

Using the assumptions outlined above, it is possible to categorize stages into average (A), good (G), and bad (B) completion quality or reservoir quality. The data can be used to highlight the sections that underperformed and may need further attention. It is beneficial to use this information to identify both the “good” and “poor” sections. For example, stages 5 through 15 generally fall below average in terms of completion and reservoir qualities. This area is the UEF section, which is characterized by lower porosity and potentially lower permeability, and apparently more depleted. In contrast, Stages 15 through 22 are above average and correspond to the Austin Chalk section. The heel section (from Stage 50 onward) predominantly underperforms, primarily due to its location within a known fault zone, leading to significant inter-well interactions with adjacent wells.

A table can be used to show above and below average completion quality (CQ) and reservoir quality (RQ) ranking for each of the stages. Color differentiation can be used. For example, gray color to indicate above average values and a blue color to indicate below average values. The lighter/darker shade of each color, and also help. For example, lighter in each group is CQ and the darker color the RQ values.

The above may illustrate an example of stage-level completion quality and reservoir quality diagnostics and evaluation from tracer production monitoring. Sample testing may be used to provide a set of data, which may be used to provide a visual indicator of wellbore performance.

### Advantages

Any tracer of embodiments herein may be selected for its particular uniqueness, and thus preferably has a different tracer characteristic (fingerprint) from other tracers used so that fluid returned to the surface may be identified. The tracer characteristic may be the chemical identity of the tracer used, such as composition or specific gravity. The tracer may be distinguishable from the tracer characteristic(s) of any other tracer used.

Any tracer of embodiments herein may be completely insoluble with the wellbore fluid(s). Any tracer may be inert in the respect that there is no effect by the tracer on the fluid and/or the formation (and/or vice versa). Any tracer (or at least a portion thereof) may have an average residence time in the target formation.

Embodiments herein may provide for a new and improved method and system related to the use of chemical tracers in various settings associated with an earthen formation, such as an oil and gas well or a geothermal well.

The tracer may be cost-effective and inert, stable at (excessively) high temperatures, compatible with formation fluids, non-intrusive to completion design, easy to use, and quickly tested. Other advantages may include use of tracers that are of a cost-effective material, inert and lightweight, easily deployed, non-hazardous and non-radioactive, a single tracer for water and oil phases, and precise sub-atomic accuracy.

The tracers may be used to provide significantly valuable wellbore performance information, especially for completion quality and/or reservoir quality, which may help a producer ascertain whether further (fracture) treatment is needed, or simply that the wellbore is unsuitable for production, regardless of any further treatment.

While preferred embodiments of the disclosure have been shown and described, modifications thereof may be made by one skilled in the art without departing from the spirit and teachings of the disclosure. The embodiments described herein are exemplary only and are not intended to be limiting. Many variations and modifications of the embodiments disclosed herein are possible and are within the scope of the disclosure. Where numerical ranges or limitations are expressly stated, such express ranges or limitations should be understood to include iterative ranges or limitations of like magnitude falling within the expressly stated ranges or limitations. The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the preferred embodiments of the present disclosure. The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

What is claimed is:

1. A method of using an inert, non-magnetic tracer in a wellbore, the method comprising:
disposing the tracer into the wellbore so that the tracer comes into contact with a target formation in communication with the wellbore;
upon contacting the target formation for an amount of time, returning a remnant fluid that includes at least a portion of the tracer to a surface;
taking a sample of the remnant fluid;
providing the sample to a surface facility;
testing the sample at the surface facility via non-destructive energy dispersive x-ray fluorescence (EDXRF) in order to analyze the remnant fluid to provide a set of data associated with performance of the wellbore,
wherein the inert, non-magnetic tracer has a first tracer composition, and
wherein the inert, non-magnetic tracer is in a solid powder form having a bulk average particle diameter of at least 0.1 μm to less than 1 μm.

2. The method of using the inert, non-magnetic tracer of claim 1, wherein prior to testing the sample, the sample is filtered.

3. The method of using the inert, non-magnetic tracer of claim 1, wherein the set of data is used to provide a visual indicator related to at least one of: completion quality, reservoir quality, and combinations thereof.

4. The method of using the inert, non-magnetic tracer of claim 1, wherein disposing the tracer into the wellbore further comprises using at least one of: using a pumpable fluid mixture, using a downhole dissolvable tool coupled with a tubestring, using a downhole dissolvable device that is not connected with any tubestring, using a perforator tool, and combinations thereof.

5. The method of using the inert, non-magnetic tracer of claim 1, the method further comprising: from the same wellbore, disposing a second non-magnetic tracer into the wellbore so that the second tracer comes into contact with a second stage of the wellbore.

6. The method of using the inert, non-magnetic tracer of claim 5, wherein the second tracer is a different composition from the tracer, but is otherwise also in non-magnetic powder form, with a respective bulk average particle diameter of at least 0.1 μm to less than 1 μm.

7. The method of using the inert, non-magnetic tracer of claim 1, wherein the target formation is associated with a frac stage, wherein the wellbore is associated with a formation temperature of at least 450° F. to no more than 2000° F., and wherein the target formation has an average permeability of 0.1 nanodarcy to 1000 nanodarcy.

8. The method of using the inert, non-magnetic tracer of claim 1, wherein no step of the method uses or is associated with at least one of: a magnet, a magnetic feature, and combinations thereof, and wherein the set of data is obtained by using an artificial intelligence neural network.

9. A method of using a tracer in a wellbore, the method comprising:
disposing the tracer into the wellbore so that the tracer comes into contact with a target formation in communication with the wellbore;
upon contacting the target formation for an amount of time, returning a remnant fluid that includes at least a portion of the tracer to a surface;
taking a sample of the remnant fluid;
providing the sample to a surface facility; and
testing the sample at the surface facility via non-destructive energy dispersive x-ray fluorescence (EDXRF) in order to analyze the remnant fluid to provide a set of fluid data associated with an elemental composition of the remnant fluid;
wherein prior to testing the sample, the sample is filtered,
wherein the tracer has a first tracer composition, and
wherein the tracer is in a non-magnetic solid powder form having a bulk average particle diameter of at least 0.1 μm to less than 1 μm.

10. The method of using the tracer of claim 9, the method further comprising:
integrating the set of fluid data with other wellbore data in order to provide a visual indicator related to performance of the wellbore; and
disposing a second tracer into the wellbore so that the second tracer comes into contact with another stage of the wellbore.

11. The method of using the tracer of claim 10, wherein the second tracer is a different composition from the tracer, but is otherwise also in non-magnetic powder form, and has a bulk average particle diameter of at least 0.1 μm to less than 1 μm.

12. The method of using the tracer of claim 11, wherein the target formation has an average permeability of 0.1 nanodarcy to 1000 nanodarcy.

13. The method of using the tracer of claim 9, wherein disposing the tracer into the wellbore further comprises using at least one of: using a pumpable fluid mixture, using a downhole dissolvable tool coupled with a tubestring, using a downhole dissolvable device that is not connected with any tubestring, using a perforator tool, and combinations thereof.

14. The method of using the tracer of claim 13, wherein no step of the method uses or is associated with at least one of: a magnet, a magnetic feature, and combinations thereof.

15. The method of using the tracer of claim 14, wherein the target formation is part of a geothermal well, and the remnant fluid is used in an energy generation process.

16. A method of using a tracer in a wellbore, the method comprising:
disposing the tracer into the wellbore so that at some point thereafter the tracer comes into contact with a first stage of the wellbore;
upon contacting the target formation for an amount of time, returning a remnant fluid that includes at least a portion of the tracer to a surface;
taking a sample of the remnant fluid;
providing the sample to a lab facility;
filtering the sample to provide a filtered sample;
testing the filtered sample at the lab facility via non-destructive energy dispersive x-ray fluorescence (EDXRF) in order to analyze the remnant fluid via energy excitation at sub-atomic level in order to provide a set of fluid data associated with an elemental composition of the remnant fluid;
from the same wellbore, disposing a second tracer into the wellbore so that at some point thereafter the second tracer comes into contact with a second stage of the wellbore,
wherein the tracer has a first tracer composition,
wherein the tracer is in a non-magnetic solid powder form,
wherein no step of the method uses or is associated with at least one of: a magnet, a magnetic feature, and combinations thereof,
wherein the second tracer is also in a non-magnetic powder form,
wherein each of the tracer and the second tracer have a respective bulk average particle diameter of at least 0.1 μm to less than 10 μm,
wherein the set of data is used to provide a visual indicator related to performance of the wellbore, and
wherein the visual indicator refers to at least one of: reservoir quality, completion quality, and combinations thereof.

17. The method of using the tracer of claim 16, wherein the respective bulk average particle diameter of each of the first tracer and the second tracer is less than 1 μm.

18. The method of using the tracer of claim 17, wherein no step of the method uses or is associated with at least one of: a magnet, a magnetic feature, and combinations thereof.
